(19) United States
(12) Patent Application Publication
(10) Pub. No.: US 2007/0048238 A1
(43) Pub. Date: Mar. 1, 2007

(54) Color cosmetic compositions and methods

(76) Inventors: Ida Marie Sandewicz, Monroe Township, NJ (US); Tatyana Zamyatin, Princeton Junction, NJ (US); Julio Gans Russ, Westfield, NJ (US); Sarah K. Jabush, Belford, NJ (US)

Correspondence Address: Julie Blackburn;Revlon Consumer Products Corporation, 237 Park Avenue, New York, NY, 10017, US

(21) Appl. No.: 11/378,681

(22) Filed: Mar. 17, 2006

Related U.S. Application Data
(60) Provisional application No. 60/712,310, filed on Aug. 30, 2005.

(51) Int. Cl. A61K 8/19; A61K 8/73

(52) U.S. Cl. 424/63; 424/70.13

(57) ABSTRACT

An anhydrous color cosmetic composition comprising a pigment component and a powder component, the improvement wherein the powder component includes microfine particle size powders in an amount sufficient to provide a composition that matches a plurality of skin shades in one, or more than one, skin tone category; and an anhydrous foundation, blush, concealer, mascara, or other cosmetic composition that has a first resting color and a second application color.

### RELATED APPLICATIONS

This application claims priority from provisional patent application serial No. 60/712,310, filed Aug. 30, 2005.

### TECHNICAL FIELD

The invention is in the field of anhydrous color cosmetic compositions that match a plurality of skin shades; develop color when applied to the skin; and methods for formulating color cosmetics that are suitable for a wide variety of skin shades so as to eliminate the number of SKUS cosmetics manufacturers must stock, as well as to reduce the resulting consumer confusion from having too many options to choose from.

### BACKGROUND OF THE INVENTION

Color matching is a common concern with respect to color cosmetic such as foundation, blush, concealer, eyeshadow, lipstick, bronzer, and essentially all color cosmetics. With respect to foundations in particular, in order to ensure that there is a suitable shade for every consumer skin color, cosmetics companies will often offer up to 30 shades in a particular brand so that all possible skin colors are represented. With color cosmetics that are accents, such as blush, eyeshadow, or concealer, there are a multiplicity of colors to choose from. The consumer is often confused about which blush or eyeshadow is suitable for her skin coloring. While the color charts depicted the colors of the various products are often quite accurate, the cosmetic may look very different on the customer's skin. The result is that the customer ends up purchasing a product that is not as flattering as it could be. She is unhappy, and that unhappiness may lead to her patronizing a different cosmetics manufacturer the next time she purchases a product in that category.

Color matching and coordinating is a particular problem in mass market outlets or self-select channels of distribution. The term “self-select” means, with respect to the outlet in which the consumer is shopping, that the consumer selects the products by herself generally without the advice and counsel of a beauty advisor. Typically self-select channels include large mass market retailers such as KMart, WalMart, or Target; or chain drug stores such as CVS, Rite-Aid, or Walgreen's. In these shopping situations, the consumer must select the proper color of foundation, blush, eyeshadow, bronzer, or concealer under conditions, such as fluorescent lighting, that may not contribute to purchase of the product that is best matched with the skin color. Particularly in mass market outlets, there are no beauty advisors to advise and counsel the purchaser. The purchaser must exercise her own judgment. As purchasers vary widely in terms of their sophistication with cosmetics, this can be a hit-or-miss proposition.

Cosmetics companies that have a substantial mass market business have tried to address this problem in a variety of ways. For example, with respect to foundation makeup, L'Oreal sells a line of foundation, blush, and concealer products under the True Match® brand. Selection of the foundation that best matches the consumer's skin color is performed in two steps. First, the shopper is asked to select the color chip that is closest to her skin color from a palette offering 10 possible choices (such as fair, light, light/medium, medium, etc). In the second step, the shopper is asked to select her skin undertone from 3 possible choices (warm, neutral, cool). The cosmetics display then indicates which of the approximately twenty four different shades are most suitable for that particular consumer. L'Oreal has recently extended the True Match® franchise to other color cosmetics such as blush and concealer. While this method of selecting color cosmetics to ensure a true match exhibits certain improvements over the traditional method of selling such products, it still requires that the consumer be capable of making interpretations that require some skill. Particularly with respect to foundation, not all consumers are sure of what their skin color is, especially when there are ten possible color chips to choose from. This decision is also made more difficult under the fluorescent lighting conditions that are often found in stores. Further complicating the decision process, is requiring the consumer to then determine skin undertone. The possibility for error in the consumer's interpretation is reasonably high, with the end result being the purchase of a foundation or other color cosmetic product that does not truly match the purchaser's skin color.

Procter & Gamble's Cover Girl brand uses a similar approach to solve the foundation matching problem with their product line Tru Blend®. The self-select shopper is asked to determine her skin tone from a palette of about 10 different shades; then determine her skin undertone (generally cool, neutral, or warm). The foundation suitable for the shopper is then identified based upon the consumer's interpretation of her skin color and undertone, and the decisions made with respect to the choices offered. The margin for error is the same as with the L'Oreal products. While consumers tend to make better color matches using the Tru Blends system, there is still a significant margin for error.

Accordingly, there is a need for simplifying the shopping experience for the consumer to ensure that the products suitable for that consumer are easily identifiable by the consumer herself, without requiring skilled interpretation or decision making. One general way to accomplish this is to reduce the number of choices the consumer is obliged to make in selecting a suitable foundation makeup, blush, bronzer, eyeshadow, concealer, mascara, lipstick, or other color product, and make the consumer interpretation required as simple as possible. For example, one obvious way to do this, is to provide three or four general categories and ask the consumer to determine what category she falls into. For example, such skin tone categories might be “light”, “light/medium”, “medium”; or “dark”. Once the consumer had made that one decision, there would be one foundation makeup or other color cosmetic product suitable for all skin shades in one skin tone category or lash colors in the one skin tone category selected. That product would be suitable on all skin shades generally found within the “light” category (e.g. foundation colors typically referred to with descriptors such as fair, ivory, buff, nude, etc.). Similarly, for products such as blush, there might be one blush suitable for use with all skin shades in one skin tone category, or possibly one blush suitable for all skin shades across all skin tone categories. In the case where the composition is a concealer, there may be one concealer that matches all skin shades in one skin tone category. In the case where the composition is an eye shadow, there may be several basic eye shadow colors that are suitable for skin shades in one, or more than one, skin tone category.

However, with respect to foundation makeup in particular, the formulation of makeup that is suitable for a wider variety of skin shades requires some skill. For example, it is important that foundation makeup match the skin color as closely as possible for obvious reasons. Generally, there are at least two factors that contribute to good matching of skin color. First is pigmenting. Mixtures of pigments will provide the basic color of the cosmetic composition. Typically this coloration is achieved by a blend of iron oxides such as black, yellow, and red. While pigments are responsible for providing the characteristic color to the foundation, they generally do not provide coverage. The term “coverage” means the ability of the color cosmetic composition to cover or conceal skin imperfections (in the case of foundation), or otherwise provide a film on the skin. Coverage is generally achieved by including ingredients in the formula that provide opacity. In most cases it is titanium dioxide or other similar powders will provide the opacity as well as to mute the effect of the pigments that are present. In general, color cosmetic compositions that have high opacity (e.g. powder content) provide greater coverage. Alternatively, color cosmetics that have lower opacity provide less coverage and a sheerer finish. While increasing the opacity of color cosmetics provides certain benefits with respect to covering skin imperfections, color cosmetics with high opacity are harder to match with skin. When manufacturers of color cosmetics sell formulas having higher opacity, they must include many more SKUS in their lines. For example, a foundation manufacturer that sells a relatively high opacity foundation may need to have 24 to 30 shades in the foundation makeup line to ensure that all the skin colors of their customer base are adequately represented. More SKUs means more expense for the cosmetics manufacturer and the retailer, and increases consumer confusion about how to select the proper foundation for her skin from the myriad of choices.

Accordingly, there is a need for color cosmetics such as foundation, blush, eyeshadow, bronzer, lipstick, concealer, and the like, that match a variety of skin tones in one shade category such that cosmetics manufacturers can reduce the number of SKUs offered for sale in their line. This will minimize cost for the manufacturer, reduce the inventory retailers must maintain, and minimize consumer confusion due to having too many options to select from.

Even more desirable are color cosmetic compositions that match a variety of skin tones or hair color, wherein such a foundation would exhibit one standard resting color (e.g. the color in the container is generally the same for all shade categories), and a second application color so that there is a consumer perception that the color cosmetic composition is “smart”, e.g. it changes color to match the keratinous surface to which the user is applying it. The color of such “smart” makeup would develop upon application to skin, giving the consumer the impression that the makeup changes color from an initial resting color to a color that matches her skin tone.

It is an object of the invention to provide a makeup composition that is more universal in its ability to match a variety of skin tones, particularly skin tones in one general category such as “light”, “light/medium”, “medium” or “dark”; or match skin tones across all categories.

It is a further object of the invention to provide color cosmetic compositions that enable cosmetics manufacturers to offer products that match the skin color variations of their customers, but with a reduced number of SKUs.

It is a further object of the invention to provide a color cosmetic composition that has a first resting color and a second application color, such that when the makeup is in a resting state (such as in the container) it exhibits a very generic color (such as white, grayish white, or bluish white), and upon application the color develops to match the skin tone of the consumer.

It is a further object of the invention to provide a color cosmetic composition that matches a plurality of skin shades in one skin tone category in order to reduce the number of SKUs sold by cosmetics manufacturers and the consumer confusion involved in trying to select the appropriate foundation, blush, eyeshadow, bronzer, concealer, lipstick, or the like from a multiplicity of shade offerings.

It is a further object of the invention to provide a color cosmetic composition that is suitable for the multiplicity of skin shades in each general skin tone category.

It is a further object of the invention to provide a color cosmetic product such as blush, concealer, eye shadow and the like, which is suitable for a multiplicity of skin shades in one skin tone category or across more than one skin tone category.

It is a further object of the invention to provide a color cosmetic product such as blush, concealer, eye shadow and the like, which exhibits a first resting color and a second application color.

### DETAILED DESCRIPTION

I. The Composition

A. Definitions

1. All percentages mentioned herein are percentages by weight unless otherwise indicated.

2. “Reactive” means, with respect to the pigment, that it is reactive upon contact with water, skin, skin fluids (such as oil or water on the skin), or hair to form color. Alternatively, reactive may also mean that the color is developed in other ways such as the mechanical agitation encountered when the cosmetic, or the pH or temperature change between the cosmetic composition in the container and after applied to skin. For example, when the composition containing the reactive pigment is applied to the skin, the mechanical agitation inherent in the application process, the water present in the skin, or the pH or temperature change, causes the pigment to develop color. In the case where the pigment is a water soluble or dispersible pigment that is dispersed or suspended in the oily phase of an anhydrous composition or anhydrous emulsion, the pigment is not exposed to water in the formula. In that case, the composition may exhibit a first resting color that is muted, or possibly a generic white or gray. However, when the water soluble or water dispersible pigment dispersed in the oily phase comes into contact with the skin, the color develops, either due to the solubilization of the pigment by the water and fluids on the skin surface, mechanical agitation which releases the pigment, or the pH or temperature change that occurs when the composition is applied to the skin.

3. “First resting color” means, with respect to the composition, that when it is in the resting state, e.g. stored in a container, it exhibits one visually distinct color.

4. “Second application color” means, with respect to the composition, that when it is applied to the skin (e.g. removed from the resting state), it exhibits a second visually distinct color that is different from the first visually distinctive color.

5. “Visually distinct” means, with respect to color differences, that they can be observed with the naked eye.

6. “Volatile” means that the ingredient has a vapor pressure of greater than about 2 millimeters of mercury at 20° C.

7. “Near volatile” means that the ingredient has a vapor pressure ranging from about 1 to 2 millimeters of mercury at 20° C.

8. “Non-volatile” means that the ingredient has a vapor pressure of less than about 1 millimeter of mercury at 20° C.

9. “SKU” means stock keeping unit, a term generally used by retailers to identify the lowest level of product detail. By way of example, a blush in the color pink, sold by a cosmetics manufacturer under the brand XY would typically have a unique numeric identifier in the form xxxx-xx, where the first four digit number would indicate blush brand XY and would be the same across all shades of brand XY, and the second two digit number after the dash would be specific to each separate shade in the brand.

10. “Skin tone” means the general color of the skin, for example, “light”, “light/medium”, “medium”, or “dark”.

11. “Skin Shade” is a particular color within a skin tone category. For example, consumers who have the skin tone “light” include those that have fair, fair/light, or light skin shades and typically wear foundation shades such as ivory, buff, or nude; or blush colors such as pink, pale rose, and so on. Consumers who have the skin tone “light/medium” have a skin color that is a cross between medium and light color and includes those that wear foundation shades such as sand beige, natural beige, or medium beige; and possibly blush colors such as rose. Consumers who fall into the skin tone category of “medium” include those that have medium colored skin and typically wear foundation shades such as honey beige, sand beige, warm or golden beige, or early tan; and blush colors such as deep rose, light wine. Consumer who fall into the general skin tone category of “deep” have dark skin and generally wear foundation shades such as mocha, caramel, etc; and possible blush shades such as deep wine, coffee, and so on. In general, skin shade is a subset of skin tone; and each skin tone category will most often include more than one skin shade.

12. The term “powder component” refers to the portion of the composition that is made up of powder ingredients when the total amount of all of the powders (such as nylon, titanium dioxide, boron nitride, mica, and so on) are added together.

13. The term “pigment component” refers to the portion of the composition that is made up of pigments when the total amount of all of the pigments (such as red iron oxide, yellow iron oxide, or black iron oxide) in the composition are added together.

B. Anhydrous

Preferably, the composition is an anhydrous composition. The term “anhydrous” means that water is not intentionally added to the composition, although small amounts of water may be present as trace ingredients in the raw materials used to make the composition. In such a case, the composition of the invention will be considered anhydrous if the water present is so little that it is not required to be listed as an ingredient on the ingredient listing for the cosmetic under the Right to Know Laws promulgated by the United States Food and Drug Administration. The composition may be in the form of an anhydrous emulsion, meaning that polar ingredients such as glycols, mono-, di-, or polyhydric alcohols, and the like are dispersed or solubilized in the oily phase of the composition.

C. Oils

The composition of the invention preferably contains one or more oils. The term “oil’ means an ingredient that is a pourable liquid at room temperature. Suitable oils may be organic, or silicone based, and suggested ranges are from about 0.1-95%, preferably from about 0.5-85%, more preferably from about 1-75% by weight of the total composition.

1. Silicone Oils

Suitable silicone oils may be volatile or non-volatile.

(a). Volatile Silicone Oils

Volatile silicones that may be used in the composition are linear or cyclic. Suitable cyclic volatile silicones have the general formula: 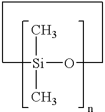
wherein n=3-6.

Examples of such cyclic volatile silicones include hexamethylcyclodisiloxane, octamethylcyclotetrasiloxane, decamethylcyclopentasiloxane, dodecamethylcyclohexasiloxane, and so on.

Preferred linear volatile silicones that may be used have the general formula:
(CH₃)₃Si—O—[Si(CH₃)₂—O]ₙ—Si—(CH₃)₃
wherein n=0-4.

Examples of such silicones include hexamethyldisiloxane (generally having a viscosity of about 0.65 centipoise), octamethyltrisiloxane (generally having a viscosity of about 1.0 centipoise), decamethyltetrasiloxane (generally having a viscosity of about 1.5 centipoise), dodecamethylpentasiloxane (generally having a viscosity of about 2.0 centipoise), and the like, with all viscosity measurements given for room temperature (25° C.). It is noted that centipoise=centistokes×specific gravity (density). As the density of such linear and cyclic volatile silicones is close to 1, then the values for both centipoises and centistokes will be essentially the same.

Linear and cyclic volatile silicones are available from various commercial sources including Dow Coming Corporation, GE Silicones, Shin-Etsu, Goldschmidt, and Wacker. Examples of suitable Dow Coming volatile silicones are those sold under the tradenames Dow Coming 244, 245, 344, and 200 fluids. Suitable volatile silicones sold by GE Silicones include SF1214, SF1528, SFE839, and the like.

(b). Non-Volatile Silicone Oils

Various non-volatile silicones may be present in the composition. Preferred non-volatile silicones have the general formula:
(CX₃)₃Si—O—[Si(CX₃)₂—O]ₙ—Si(CX₃)₃
wherein n is 5 or greater, preferably from about 5 to 1,000,000; and each X is independently H, phenyl, trimethylsiloxy, fluoro, or C₁₋₁₀ alkoxy. Examples of such silicone oils include those referred to as dimethicone, phenyl trimethicone, diphenyl dimethicone, and the like.
wherein n is 5 or greater, preferably from about 5 to 1,000,000; and each X is independently H, phenyl, trimethylsiloxy, fluoro, or C₁₋₁₀ alkoxy. Examples of such silicone oils include those referred to as dimethicone, phenyl trimethicone, diphenyl dimethicone, and the like.

2. Organic Oils

The composition of the invention may also contain one or more organic oils. Suitable organic oils include mono-, di-, or triesters, paraffinic hydrocarbons, and the like.

(a). Paraffinic Hydrocarbons

The composition may contain one or more paraffinic hydrocarbons that may be volatile or non-volatile.

(i). Volatile Paraffinic Hydrocarbons

Suitable volatile paraffinic hydrocarbons include those having straight or branched chains having about 5 to 18 carbon atoms, more preferably about 8-18 carbon atoms. Examples include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of about 70-225, preferably about 160 to 190 and a boiling point range of about 30 to 320, preferably 60-260° C., and a viscosity of less than about 10 centipoise at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable.

(ii). Near Volatile or Non-Volatile Paraffinic Hydrocarbons

It may be desired to include one or more near volatile or non-volatile paraffinic hydrocarbons in the composition. Examples of such hydrocarbons include straight or branched chain hydrocarbons having from 18 to 40 carbon atoms such as heneicosane, docosane, n-octadecane, nonadecane, eicosane, tetracosane, pentacosane, hexacosane, heptacosane, octacosane, dotriacontane, tritriacontane, hexatriacontane, hydrogenated polyisobutene, mineral oil, pentahydrosqualene, squalene, squalane, and so on.

(b). Esters

(i). Monoesters

Suitable monoesters are generally formed by the reaction of a monocarboxylic acid and an aliphatic alcohol that may be substituted with one or more substituents such as hydroxyl, alkyl, or alkoxy groups. Such esters preferably have the formula R—COOH wherein R is a C₁₋₄₅ straight or branched chain, saturated or unsaturated alkyl, alkoxy, C₁₋₃₀ alkoxy alkyl, and the like, any of which such mentioned substituents may be substituted with hydroxyl, C₁₋₃₀ alkyl, or C₁₋₃₀ alkoxy groups. Examples of such monoesters include monoesters of fatty acids having from 6 to 30 carbon atoms, such as stearic acid, malic acid, oleic acid, linoleic acid, behenic acid, palmitic acid, myristic acid, and so on. Further examples of monoesters include isostearyl malate, isopropyl palmitate, stearyl stearate, isopropyl malate, hexyl laurate, cetyl isononanoate, butyl oleate, cetyl palmitate, hexadecyl octanoate, and so on.

(ii). Diesters

Suitable diesters that may be used in the compositions of the invention are the reaction product of a dicarboxylic acid and an aliphatic or aromatic alcohol, or alternatively, the reaction product of a monocarboxylic acid and an aliphatic or aromatic alcohol having at least two hydroxyl groups. The dicarboxylic acid or the alcohol may contain from 2 to 45 carbon atoms, and may be in the straight or branched chain, saturated or unsaturated form. In the case where the ester is formed from a dicarboxylic acid, it may be subsituted with one or more hydroxyl groups. The aliphatic or aromatic alcohol may also contain from 2 to 30 carbon atoms, and may be in the straight or branched chain, saturated, or unsaturated form. The aliphatic or aromatic alcohol may also be substituted with one or more substituents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl adipate, disostearyl fumarate, and so on.

(iii). Triesters

Suitable triesters that may be used in the compositions include those that are the reaction product of a tricarboxylic acid and an aliphatic or aromatic alcohol, or the reaction product of a mono- or dicarboxylic acid and an aliphatic alcohol having two, three, or more substituted hydroxyl groups. As with the mono- and diesters mentioned above, either the acid or the alcohol or both may contain from about 2 to 30 carbon atoms, and may be saturated or unsaturated, straight or branched chain, and may be substituted with one or more hydroxyl groups. Preferably, one or more of the acid or alcohol is a fatty acid or alcohol containing from about 6 to 30, preferably from about 14 to 22 carbon atoms. Examples of triesters include triarachidin, tributyl citrate, tri C₁₂-₁₃ alkyl citrate, tricaprylin, tricaprylyl citrate, tridecyl behenate, trioctyldodecyl citrate, tridecyl behenate, tridecyl cocoate, tridecyl isononanoate, triisostearyl citrate, and so on.

D. Particulates

The composition of the invention comprises particulates, which include both pigments and powders. The term “powder” refers to white particulates (such as titanium dioxide) or non-pigmented particulates (such as boron nitride, nylon, etc.), that are used for muting color or, with respect to color cosmetics such as foundation or blush, used for providing opacity or coverage. Preferred compositions of the invention comprise both pigments and powders. Suggest ranges of pigment are from about 0.001-90%, preferably from about 0.005-85%, more preferably from about 0.01-75% by weight of the total composition. Suggested ranges of powders are from about 0.001-90%, preferably from about 0.005-80%, more preferably from about 0.01-70% by weight of the total composition.

1. Pigments

Suitable pigments include inorganic or organic pigments. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments also generally consist of insoluble metallic salts of certified color additives, referred to as the Lakes.

Inorganic pigments include iron oxides such as red, black, yellow and the like; ultramarines, chromium, chromium hydroxide colors, and mixtures thereof.

Pigments used in the composition of the invention may be regular sized, ranging from 35-200, from about 40 to 100 microns, or such pigments may be in the microfine particle size form, where the particle sizes range from about 0.05-34 microns, preferably from about 0.1-25 microns, more preferably from about 1-25 microns, most preferably from about 1-15 microns in diameter.

In one preferred embodiment of the invention the pigments are either inherently hydrophilic or are coated with a substance that will cause the pigment to be water or oil dispersible. While such pigments are incorporated into an anhydrous composition or anhydrous emulsion, they are water soluble or dispersible even when dispersed in the nonpolar phase of the anhydrous composition. Most preferred is where the pigments present in the composition are water dispersible and reactive. Thus, such pigments may be dispersed in the oily or particulate phase of the anhydrous composition or anhydrous emulsion, while the composition is in the resting state. The dispersion of the pigments in the anhydrous composition provides a composition that preferably does not match the skin because the full color of the pigments is not visible when suspended in the composition. The color of the composition in the resting state when the pigments are suspended in the composition and not in contact with water or other fluids, may be white, grayish white, whitish blue, or a variety of other colors depending on the color of the powders or other non-reactive pigments that are used in the composition. However, when the composition is applied to skin, the reactive pigments come into contact with the skin, may react with the water and other fluids on the skin, or due to mechanical agitation, pH or temperature change, cause the color of the composition to develop directly on the skin when it is applied. The development of the color directly on the skin from a non-skin matching color to a skin matching color gives the consumer the impression that the composition is “smart” and capable of changing color to exactly match her skin tone, or lash or brow color if the composition is a mascara, eyeliner, or brow color.

If the pigments are coated with a substance in order to make them dispersible in the anhydrous composition or emulsion, a variety of materials are suitable including polysaccharides, carbohydrates, or biological polymers.

Examples of biological polymers suitable as pigment coating materials include those having repeating monomers that are generally of the carbohydrate type. Included are materials obtained from edible grains such as corn starch; or chemically modified materials obtained from edible grains. Examples of such biological polymers include, but are not limited to, biosaccharide gum; chitosan and derivatives thereof such as butoxy chitosan, carboxymethyl chitosan, carboxybutyl chitosan, chitosan gluconate, chitosan adipate, chitosan glycolate, chitosan lactate, etc; chitin and derivatives thereof such as carboxymethyl chitin, chitin glycolate; cellulose and derivatives thereof such as cellulose acetate; microcrystalline cellulose; distarch phosphate; sodium hyaluronate; soluble proteoglycan; galactoarabinan; glycosaminoglycans; glycogen; sclerotium gum; dextran; starch and derivatives thereof, and the like.

Examples of carbohydrates suitable for use in coating pigments may be described as polyhydroxy aldehydes or ketones that conform to the general structure:
Cₓ(H₂O)_{y}
wherein x and y range from about 1 to 1,000,000. The carbohydrates may be monosaccharides, disaccharides, or polysaccharides. Examples of suitable carbohydrates include those set forth on pages 1660 through 1662 of the International Cosmetic Ingredient Dictionary and Handbook, Volume 2, Eighth Edition, 2000, which is published by The Cosmetic, Toiletry, and Fragrance Association (C.T.F.A.), hereby incorporated by reference in its entirety. Such carbohydrates include, but are not limited to, amylodextrin, betaglucan, cyclodextrin, modified corn starch, glycogen, hyaluronic acid, hydroxypropyl cyclodextrin, lactose, maltitol, guanosine, glyceryl starch, triticum vulgare starch, trehalose, sucrose or derivatives thereof; raffinose, sodium chondroitin sulfate, and the like.

Also suitable for treating the pigments are C₁₋₂₀ alkylene glycols or C₁₋₂₀ alkylene glycol ethers either alone or in combination with tri-C₁₋₂₀ alkylsilanes with the alkyl groups ranging from about C₁₋₂₀. Examples of such pigments include PEG alkyl ether alkoxy silane treated pigments including but not limited to PEG-8 methyl ether triethoxysilane treated pigments sold by Kobo under the brand name “SW” pigments.

Also suitable are-pigments treated with silicones such as dimethicones having hydrophilic groups also known as dimethicone copolyols or alkyl dimethicone copolyols. In particular, such dimethicones include those having C₁₋₂₀ alkylene oxide repeating units such as ethylene, propylene, and the like. One particular suitable pigment is PEG-12 dimethicone treated pigment sold by Sensient Corporation under the trade name LCW AQ pigments.

Another suitable pigment includes microencapsulated pigments such as iron oxides where the capsule material includes polymeric materials. One particularly suitable example is iron oxide micro-pigments sold by Tagra, Netanya, Israel and distributed by TRI-K Industries under the trade name TAGRA. One type of Tagra cap includes microencapsulated yellow iron oxide, titanium dioxide, boron nitride, ammonio methacrylate copolymer, and triethyl citrate, sold as YellowCap 1.

Particularly preferred is where the makeup contains iron oxide pigments, which are coated with galactoarabinan, which is a polysaccharide obtained from the extraction of the larch tree Larix. Such pigments are available from Color Techniques under the GA series. For example, a suitable red iron oxide is GA-7293 hydrophilic red iron oxide sold by Color Techniques.

2. Powders

The composition also contains powders or fillers that are present for adjusting the color of the composition, and in some cases may provide a sunscreen effect by physical blocking of UV radiation. Preferably, the particle size of the powders ranges from 0.05 to 100 microns, and are present in ranges of 0.001-90%, preferably 0.005-80%, more preferably from about 0.01-70% by weight of the total composition.

Examples of powders include white or non-pigmentitious powders such as titanium dioxide, bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, chalk, hydrated silica, kaolin, magnesium aluminum silicate, magnesium trisilicate, montmorillonite, talc, mica, zinc laurate, zinc myristate, zinc rosinate, nylon, silica silylate, silk powder, sericite, tin oxide, titanium hydroxide, trimagnesium phosphate, walnut shell powder, and the like. While titanium dioxide is commonly considered to be a white pigment when used in paints, in color cosmetic compositions it is used more for its ability to mute color, and/or provide an opaque or semi-opaque finish, or provide sunscreen protection, then as a colorizing ingredient. The above mentioned powders may be surface treated with lecithin, amino acids, mineral oil, silicone oil or various other agents either alone or in combination, which coat the powder surface and render the particles more lipophilic in nature.

Most preferred is where the powder component of the composition contains a portion of powders in the microfine particle size form so as to minimize the opacity of the resulting composition and thereby enable the composition to match plurality of skin shades in one skin tone category or across all skin tones. Preferred microfine particle sizes range from about 0.1-30 microns, more preferably from about 0.5-20 microns, most preferably from about 1-17 microns in diameter. In the most preferred embodiment of the invention, at least a portion of the titanium dioxide present in the formula will be in the ultrafine form; or more preferably from about 0.1-15%, preferably from about 0.5-10% by weight of the total composition will be microfine particle titanium dioxide; with the remaining amount of titanium dioxide (ranging from about 0.1-5% by weight-of the total composition) being of a regular particle size, ranging from about 35-100 microns. It is noted that typical foundation makeup compositions generally contain from about 7 to 15% of titanium dioxide powder, with such titanium dioxide generally being of a standard particle size of about 50 microns. This provides the resulting color cosmetic composition with an appreciable degree of opacity, which is excellent for covering skin imperfections. In the color cosmetic compositions of the invention, the substitution of ultrafine particle size powders for regularly sized particles will provide a composition with substantially reduced opacity. This will provide a makeup that provides a more sheer finish on the skin, which in turn ensures that a single foundation makeup formula will more easily match a plurality of skin tones. The pigments present in the composition will accent the color of the skin, and if they are microfine pigments will tend to do so in a very subtle manner. At the same time, the microfine powders present will mute the pigments to provide the desired skin tone, but will not provide an amount of opacity that will compromise the versatility of the composition in matching a variety of skin tones. Additionally, the microfine powder particles will contribute to the resting color of the composition, so that the most preferred embodiment of a foundation composition appears white, whitish gray, or possibly whitish blue while in the resting state, but upon application to the skin the reactive pigments react with the water present on the skin surface to develop color that matches or co-ordinates with the skin tone of the individual. In the case where the reactive pigments are made hydrophilic by coating with a hydrophilic substance such as galactoarabinan, upon contact with the skin surface, the coating or shell dissolves upon contact with skin water and fluids. The dissolution of the shell frees the pigment particle, which causes the color to become more visible.

The microfine particle size powders may confer an additional benefit of providing SPF to the composition. For example, very fine particle zinc oxide and/or titanium dioxide may cause the composition to exhibit SPF (sun protective factor) values as high as 5 to 25. For example, if the composition contains from about zinc oxide, titanium-dioxide, or mixtures thereof having particle sizes ranging from about 0.005 to 10 microns, the composition will likely have SPF values ranging from 5 to 25.

Further, it is noted that in general, the powder component (or the portion of powders in the formula) in a typical foundation makeup generally ranges from about 5 to 30% by weight of the total foundation composition. In order to provide a foundation that better matches a plurality of skin shades in one skin tone category, a portion of such powders in the powder component must be found in the microfine particle size range (preferably the majority of particles in the 1-17 micron size range). With respect to foundation or concealer, preferred is where about 35-100%, preferably from about 40-95%, or parts by weight, of the total powder component comprises powders in the microfine particle size range. Most preferred is where the powder component present in the foundation contains from about 45-95% or parts by weight of the total powder component of microfine titanium dioxide as the powder component. It has been found that where at least 35-100% by weight of the total powder component is microfme, or where from about 30-95% by weight of the total titanium dioxide content is in the microfine particle size form, the resulting foundation will match a plurality of skin shades in one skin tone category.

E. Mono-, Di-, or Polyfunctional Organic Alcohols

It may be desirable to include one or more mono-, di-, or polyfunctional organic alcohols in the composition. If present, such alcohols may range from about 0.001-25%, preferably from about 0.005-20%, more preferably from about 0.01-15% by weight of the total composition. It is particularly desired to include such alcohols in the composition when it is in the form of an anhydrous emulsion.

Such alcohols may function as non-aqueous solvents, humectants, astringents, or similar. Preferably, such alcohols have the general formula R—OH, where R is a C₂₋₁₀ straight or branched chain alkyl that may be unsubstituted or substituted with one or more hydroxyl groups; a C₂-₁₀ alkyl amido alkyl, or a C₂₋₁₀ alkyl amido alkoxy. Preferably R has one or more substituted hydroxyl groups, making R—OH polyhydric. The alcohol may also be a dispersant for one or more ingredients in the formula.

Examples of suitable alcohols include isopropanol; ethanol; alkaline glycols such as ethylene, propylene, or butylene glycol; glycerin; panthenol; panthenyl ethyl ether; phytantriol; or mixtures thereof.

Panthenol is a trihydric alcohol of the formula R—OH wherein R is an alkyl amido alkyl having two substituted hydroxyl groups. It has the formula:

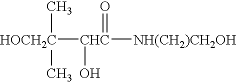

Panthenyl ethyl ether, a monohydric alcohol, is the ethyl ether of panthenol having the formula:

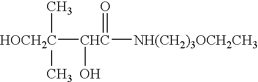

Phytantriol is an aliphatic polyhydric alcohol having the general formula:

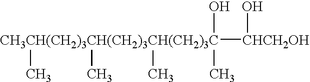

F. Surfactants

Even though the composition is anhydrous, it may contain one or more surfactants. Such surfactants may have film forming properties or aid in the suspension of pigments or other ingredients that have hydrophilic moieties. If present, suggested ranges of surfactant may be from about 0.001-45%, preferably from about 0.005-35%, more preferably from about 0.01-10% by weight of the total composition. Suitable surfactant may be organic or silicone based.

1. Silicone Surfactants

Preferred nonionic silicone surfactants include those having at least one hydrophilic radical and at least one lipophilic radical. These silicone surfactants may be a liquid or solid at room temperature and are water-in-oil or oil-in-water type surfactants that have a Hydrophile/Lipophile Balance (HLB) of about 2 to 18. Preferably the silicone surfactant is a nonionic surfactant having an HLB of about 2 to 12, preferably about 2 to 10, most preferably about 4 to 6. The HLB of a nonionic surfactant is the balance between the hydrophilic and lipophilic portions of the surfactant and is calculated according to the following formula:
HLB=7+11.7×log M_{w}/M₀
where M_{w} is the molecular weight of the hydrophilic group portion and M₀ is the molecular weight of the lipophilic group portion.

The polymeric silicone surfactant used in the composition may have any of the following general formulas:
MₓQ_{y}, or
MₓT_{y}, or
MDₓD′_{y}D″_{z}M

wherein:

each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof;

T is a trifunctional siloxy unit having the empirical formula R′SiO_{1.5} or RSiO_{1.5} wherein R is methyl and R′ is a C₂₋₂₂ alkyl or phenyl.

Q is a quadrifunctional siloxy unit having the empirical formula SiO_{4/}2; and

D, D′, D″, x, y, and z are as set forth below, with the proviso that the compound contains at least one hydrophilic radical and at least one lipophilic radical. Preferred is a linear silicone of the formula:
MDₓD′_{y}D″_{z}M
wherein M=RRRSiO_{1/2}
D=RR SiO_{2/2}
D′=RR′SiO_{2/2}
D″=R′R′SiO_{2/2}
x, y, and z are each independently 0-1000,
where R is methyl or hydrogen, and R′ is a hydrophilic radical or a lipophilic radical, with the proviso that the compound-contains at least one hydrophilic radical and at least one lipophilic radical.
   Most preferred is wherein
M=trimethylsiloxy
D=[Si(CH₃)₂—O]_{2/2}
D′=Si[(CH₃)][(CH₂)ₙCH₃]O_{2/2} where n=0-40,
D″=Si[(CH₃)][(CH₂)ₒ—O—PE)]O_{2/2} where PE is (—C₂H₄O)ₐ(—C₃H₆O)_{b}H, o=40,
a=1-100 and b=1-100, and

More specifically, suitable silicone surfactants have the formula: 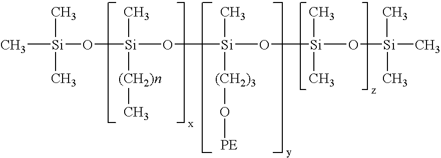
wherein n is 0-40, preferably 12-18, most preferably 14; and
PE is (—C₂H₄O)ₐ(—C₃H₆O)_{b}—H
where x, y, z, a, and b are such that the maximum molecular weight of the polymer is approximately 50,000. An example of such a silicone surfactant is where n=14, having the C.T.F.A. name cetyl dimethicone copolyol. Cetyl dimethicone copolyol may be referred to more specifically by enumerating the number of repeating ethylene oxide and propylene oxide units in the polymer. For example, a particularly suitable cetyl dimethicone copolyol for use in the invention is cetyl PEG/PPG-10/1 dimethicone having 10 PEG units for every 1 PPG unit.

Another type of silicone surfactant that may be used in the compositions of the invention are emulsifiers sold by Union Carbide under the Silwet™ trademark, which are referred to by the C.T.F.A. name dimethicone copolyol. One type of dimethicone copolyol may be more specifically referred to as PEG/PPG 18/18 dimethicone, which is dimethicone having 18 PEG units and 18 PPG units on the EO (ethylene oxide)/PO (propylene oxide) substituent.

Also suitable as nonionic silicone surfactants are hydroxy-substituted silicones such as dimethiconol, which is defined as a dimethyl silicone substituted with terminal hydroxy groups.

Examples of suitable silicone surfactants are those sold by Dow Coming under the tradename Dow Coming 3225C or 5225C Formulation Aid, Dow Coming 190 Surfactant, Dow Coming 193 Surfactant, Dow Coming Q2-5200, and the like are also suitable. In addition, surfactants sold under the tradename Silwet by Union Carbide are also suitable. Preferred silicone surfactants for use in the compositions of the invention are dimethicone copolyol or cetyl dimethicone copolyol.

2. Organic Surfactants

The composition may contain one or more organic surfactants either in lieu of, or in combination with, the silicone surfactants mentioned above.

(a). Alkoxylated Alcohols or Ethers

Examples of nonionic organic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Steareth 2-100, formed by the reaction of stearyl alcohol and ethylene oxide where the number of repeating ethylene oxide units ranges from 2 to 100; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, where the number of repeating ethylene oxide units is 1 to 45; laureth 1-100 formed by the reaction of lauryl alcohol and ethylene oxide where the number of repeating ethylene oxide units is 1 to 100; and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol, are also suitable.

(b). Alkoxylated Carboxylic Acids

Also suitable surfactants are alkyoxylated carboxylic acids, which are formed by the reaction of a carboxylic acid with an alkylene oxide or with a polymeric ether. The resulting products have the general formula: 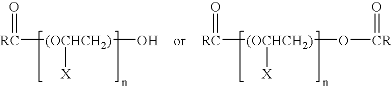
where RCO is the carboxylic ester radical, X is hydrogen or lower C₁₋₄ alkyl, and n is the number of polymerized alkoxy groups. In the case of the diesters, the two RCO-groups do not need to be identical. Preferably, R is a C₆₋₃₀ straight or branched chain, saturated or unsaturated alkyl, and n is from 1-100.

(c). Monomeric or Polymeric Ethers

Suitable surfactants also include monomeric, homopolymeric or block copolymeric ethers. Such ethers are formed by the polymerization of monomeric alkylene oxides, generally ethylene or propylene oxide. Such polymeric ethers have the following general formula: 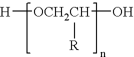
wherein R is H or lower C₁₋₄ alkyl and n is the number of repeating monomer units, and ranges from 1 to 500.

(d). Sorbitan Derivatives

Other suitable nonionic surfactants include derivatives of sorbitan, for example form by the alkoxylation of sorbitan, or by the reaction of C₁₋₂₅, preferably C₆₋₂₀ carboxylic acids with sorbitol or hexitol anhydrides derived from sorbitol.

For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85.

Examples of sorbitan derivatives include the reaction product of sorbitol or the hexitol anhydrides thereof with fatty acids to form derivative such as sorbitan oleate, sorbitan palmitate, sorbitan sesquiisostearate, sorbitan stearate, sorbitan sesquioleate, and so on.

G. Waxes

It may be desirable to include one or more waxes in the composition to increase viscosity, provide stability, or for other functional purposes. If present, suggested ranges of such waxes are from about 0.01-45%, preferably 0.05-35%, more preferably from about 0.1-25% by weight of the total composition. Such waxes may be solid or semi-solid at room temperature. The waxes preferably have a melting point of about 39 to 135° C., preferably in the range of 45 to 95° C., most preferably 55 to 95° C.

Suitable waxes generally include animal waxes, plant waxes, mineral waxes, silicone waxes, synthetic waxes, and petroleum waxes. More specifically, these waxes include tribehenin, bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEG-20 sorbitan beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, stearoxy dimethicone, dimethicone behenate, stearyl dimethicone, and the like, as well synthetic homo-and copolymer waxes such as PVP/eicosene copolymer, PVP/hexadecene copolymer, and the like. Particularly preferred is where the wax is an organic wax, tribehenin.

H. Stabilizers

The composition of the invention may contain one or more ingredients that stabilize the composition, particularly if the composition is in the anhydrous emulsion form. If present, suggested ranges of stabilizers are from about 0.001-10%, preferably from about 0.005-8%, more preferably from about 0.01-3% by weight of the total composition. Suitable emulsion stabilizers include alkali metal or alkaline earth metal salts such as magnesium sulfate, sodium chloride, magnesium chloride and the like; or EDTA, HEDTA or derivatives thereof. Preferred emulsion stablizers include sodium chloride, tetrasodium EDTA, or mixtures thereof.

I. Film Forming Ingredients

If desired, the composition may contain one or more film forming ingredients. If present, the film former may range from about 0.1-45%, preferably about 0.5-20%, more preferably about 1-15% by weight of the total composition. Suitable film formers include resinous plant extracts, synthetic polymers, and the like.

1. Resinous Plant Extracts

Examples of resinous plant extracts that provide film forming properties include materials such as rosin and shellac, or derivative thereof.

2. Synthetic Polymeric Film Formers

Suitable synthetic polymers may be silicone or organic based. Particularly preferred are siloxy silicate polymers having the following general formula: 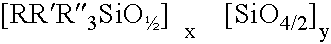
wherein R, R′ and R″ are each independently a C₁₋₁₀ straight or branched chain alkyl or phenyl, and x and y are such that the ratio of (RR′R″)₃SiO_{1/2} units to SiO₂ units ranges from about 0.5 to 1 to 1.5 to 1.

Preferably R, R′ and R″ are each a C₁₋₆ alkyl, and more preferably are methyl and x and y are such that the ratio of (CH₃)₃SiO_{1/2} units to SiO_{4/2} units is about 0.75 to 1. Most preferred is this trimethylsiloxy silicate containing 2.4 to 2.9 weight percent hydroxyl groups, which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Coming Corporation under the tradename 749 Fluid, which is a blend of about 40-60% volatile silicone and 40-60% trimethylsiloxy silicate. Dow Coming 749 Fluid, in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41.

Also suitable are synthetic polymers such as homo- or copolymers of monomers such as acrylic acid, methacrylic acid or C ₁₋₃₀ esters of acrylic or methacrylic acid, vinyl pyrrolidone, vinyl acetate, urethane, C₁₋₃₀ hydroxy esters of acrylic or methacrylic acid, vinyl isodecanoate, styrene, and olefins such as ethylene, propylene, butene, pentene, decene, hexadecene, and so on.

J. Finish Enhancers

The claimed compositions contain one or more compounds that enhance the finish, or aesthetic properties, of the composition after it is applied to skin. Preferred finish enhancers are synthetic elastomers, which may be silicone elastomers or organic polymers having elastomeric properties. The term “elastomer” means a compound exhibits properties associated with rubber such as extensibility with applied force, retractibility upon release of the force, and lack of permanent deformation as a result of extension. Rubber like properties are generally seen in high molecular weight cross-linked polymers having weak intermolecular forces. If present, suggested ranges are from about 0.001-20%, preferably from about 0.005-15%, more preferably from about 0.01-10% by weight of the total composition.

Preferred elastomers are generally in the solid particulate form having particle size ranging from about 0.05 to 75 microns. Elastomers will often provide a velvety smooth finish to the composition, improved spreadability and blendability, and a light, non-greasy feel.

1. Synthetic Organic Polymeric Elastomers

A variety of cross-linked synthetic organic polymeric elastomers may be used as finish enhancers, including those polymerized from various types of ethylenically unsaturated monomers such as acrylic acid, methacrylic acid, and simple esters thereof, vinyl monomers such as vinyl acetate, vinyl isodecanoate, methyl vinyl ether; maleic anhydride. These monomers may be copolymerized with one or more organic compounds such as esters, glycols, fatty acids, and so on. Examples of such polymers include acrylates/VA crosspolymer, acrylates/vinyl isodecanoate crosspolymer, adipic acid/diethylene glcyol/glycerin crosspolymer, allyl methacrylates crosspolymer, HDI/trimethylolhexyllactone crosspolymer, lauryl acrylate/VA crosspolymer, methyl methacrylate crosspolymer, PVM/MA decadiene crosspolymer, PEG crosspolymer, PPG-35/PPG-51 glyceryl ether/IPDI crosspolymer, trimethyl pentanediol/adipic acid/glycerin crosspolymer, and so on. Particularly preferred is HDI/trimethylolhexyllactone crosspolymer which is a crosslinked condensation polymer formed from the reaction of hexyldiisocyanate with the esterification product of trimethylolpropane with 6 to 7 moles of hexyllactone. This polymer is available from Kobo Products under the tradename BPD-500 which is a combination of silicate and the polymer having the C.T.F.A. name HDI/trimethylol hexyllactone crosspolymer (and) silica. It is a fine white powder having a particle size of about 5-20 microns comprising about 95-99% polymer and 1-5% silica.

2. Silicone Elastomers

Also suitable for use as finish enhancers are silicone elastomers such as those disclosed in U.S. Pat. No. 6,171,581, which is hereby incorporated by reference in its entirety. Examples of such elastomers include cetearyl dimethicone/vinyl dimethicone crosspolymer, dimethicone copolyol crosspolymer, dimethicone crosspolymer, dimethicone/phenyldimethicone crosspolymer, dimethicone/vinyl dimethicone crosspolymer, and mixtures thereof.

K. Botanical Ingredients

The claimed composition may contain one more botanical ingredients that are derived from plants, flowers, herbs, and the like. If present, such botanicals may range from about 0.001-10%, preferably from about 0.005-8%, more preferably from about 0.01-5% by weight of the total composition.

Suitable botanicals include extracts of aloe, gingko biloba, panax ginseng, camellia sinensis, centaurea cyanus flower extract, grape seed extract, and the like.

L. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D, C, and K, as well as derivatives thereof are suitable. Particularly preferred are derivatives of vitamins C, E, and A such as magnesium ascorbyl phosphate, retinyl palmitate, tocopheryl acetate, and mixtures thereof.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

M. Other Ingredients

A variety of other ingredients may be used in the composition, such as fragrances, preservatives, alpha or beta hydroxy acids, or mixtures thereof. A suggested range for the totality of such ingredients is about 0.001 to 3% based on the total weight of the composition. Typical preservatives include methyl, ethyl, and propyl parabens and phenoxyethanol. Suitable alpha or beta hydroxy acids include glycolic, malic, lactic, and salicylic acids.

N. Makeup Shades, Skin Tones, Color Categories

Set forth below is a table identifying certain commercially available makeup brands and shades, and listing shades that are considered to fall within the various skin tone or general color ategories listed. It is noted that in some cases the various brands do not sell products in all skin tone categories. For example, in many brands there are no shades that fall into the “dark” category, meaning that the cosmetic manufacturer does not offer shades suitable for consumers who have a skin tone falling in that category.

| | | | | Total Titanium |
|---|---|---|---|---|
| Skin Tone or | | | Total Powder | Dioxide |
| General Color | | | Content wt % | Content wt % |
| Category | Shade | Brand | (range) | (range) |
| Light | Translucent | Almay Clear | 20-80 | 5-30 |
| | Ivory | Complexion | | |
| Light/medium | Sand | Light & Perfect | | |
| Medium | Beige | Pressed Powder | | |
| Light | Translucent | Almay Time Off | 8-85 | 4-30 |
| | Ivory | Age Smoothing | | |
| Light/Medium | Sand | Pressed Powder | | |
| Medium | Beige | | | |
| Natural | Natural Elements | Almay Stay | 20-90 | 20-80 |
| | Twilights | Smooth Beyond | | |
| Dark | Coffee Break | Powder | | |
| Shimmery | Shimmers | Eyeshadow | | |
| Pinks | Petal | Almay Stay | 10-90 | 5-80 |
| | Misty Rose | Smooth Beyond | | |
| | Mauve Shimmer | Powder Blush | | |
| | Blushing Pink | | | |
| | Peach Shimmer | | | |
| Berries | Berry | | | |
| | Plum Toasty | | | |
| Bronzer | All Over | | | |
| | Bronzer | | | |
| Light | Fair | Almay Powder | 10-90 | 5-70 |
| | Light | Skin Stays Clean | | |
| Medium | Medium | | | |
| Deep | Deep | | | |
| Light | Nearly Nude | Almay Nearly | 5-40 | 2-35 |
| | Nearly Naked | Naked Touch- | | |
| Light/Medium | Nearly Sand | Pad Liquid | | |
| | Nearly Beige | Makeup | | |
| Medium | Nearly Warm | | | |
| | Nearly Honey | | | |
| Pinks | Bark Pink | Almay Nearly | 20-90 | 5-70 |
| | Peach Au | Naked Touch- | | |
| | Naturel | Pad Blush | | |
| Berries | Berry Nude | | | |
| Dark | Revealing Raisin | | | |

| | | | | Titanium |
|---|---|---|---|---|
| Skin Tone or | | | Powder Content | Dioxide |
| General Color | | | wt % | Content wt % |
| Category | Shade | Brand | (range) | (range) |
| Neutrals | Neutral Khaki | Revlon Colorstay | 20-95 | 5-30 |
| | Sandstorm | 12 Hour | | |
| | In the Buff | Eyeshadow | | |
| Rose | Sterling Rose | | | |
| Wines | Blushed Wine | | | |
| | Berry Bloom | | | |
| Deep | Copper Spice | | | |
| | Coffee Bean | | | |
| Blue/Green | Stonewash | | | |
| | Denim | | | |
| | Spring Moss | | | |
| Light | Ivory Beige | Revlon Age- | 5-60 | 2-25 |
| | Nude Beige | Defying Makeup | | |
| Light/Medium | Sand Beige | and Concealer | | |
| | Natural Beige | with Botafirm | | |
| | Medium Beige | | | |
| Medium | Honey Beige | | | |
| | Natural Tan | | | |
| | Early Tan | | | |
| Light | Ivory Beige | Revlon New | 6-50 | 2-19 |
| | Tender Peach | Complexion | | |
| Light/Medium | Sand Beige | One-Step | | |
| | Natural Beige | Compact | | |
| | Medium Beige | Makeup | | |
| Medium | Honey Beige | | | |
| | Warm Beige | | | |
| | Toast | | | |
| | Natural Tan | | | |
| Dark | Rich Tan | | | |
| | Caramel | | | |
| | Cool Beige | | | |

The above commercial foundation, blush, and eyeshadow products contain a powder component that includes titanium dioxide. Many of the brands noted exhibit a multiplicity of different shades for each tone category. If microfine particles are used for a portion of the powder component, more preferably if microfine titanium dioxide particles are used for a portion of the titanium dioxide component, in accordance with the invention, the resulting composition will have a more universal character, e.g. will match to a wider variety of skin shades in one skin tone category or across skin tone categories.

It is further noted that the composition and method of the invention are also suitable for products such as blush, concealer, and the like. For example, there is a commercial advantage in providing formulas for blush where only a few blush colors may be offered and those colors are more universal among a wide variety of skin shades. Rather than selling 15-20 different SKUs of blush, all having a different shade, the cosmetics manufacturer may use the technology of the invention to provide 1 to 5 shades that may be used across all different skin shades in the various skin tone categories or across all skin tone categories.

Similarly, typically cosmetics manufacturers offer concealers in light, medium, or dark shades. The compositions and methods of the invention will enable the cosmetics manuafacturer to even reduce the number of concealer offerings to one or two.

The compositions and methods of the invention may also be used for lipcolor, eyeshadow, and a variety of other cosmetic products where it is desired to reduce the number of SKUs by providing more a more universal color suitable for skin shades across a variety of skin tone categories; and/or where it is desirable to have the cosmetic composition develop color on the skin when applied in order to provide a composition that appears “smart”.

The invention will be further described in connection with the following examples, which are set forth for purposes of illustration only.

### EXAMPLE 1

An anhydrous foundation makeup compositions were made as follows:

| Seq | Ingredient | w/w % | |
|---|---|---|---|
| 1 | Titanium dioxide, cyclomethicone, | 15.00 | 15.00 |
| | dimethicone copolyol | | |
| 1 | Titanium dioxide, methicone | 4.20 | 4.20 |
| 1 | Zinc oxide, methicone | 2.20 | 2.20 |
| 1 | Boron nitride | 1.50 | 1.50 |
| 1 | Mica, methicone | 4.30 | 4.30 |
| 1 | Aloe Vera Powder | 0.01 | 0.01 |
| 1 | Silica | 1.00 | 1.00 |
| 1 | Bismuth oxychloride | 0.95 | 0.95 |
| 1 | HDI/trimethylolhexyllactone crosspolymer | 0.95 | 0.95 |
| 1 | Lauroyl lysine | 2.10 | 2.10 |
| 1 | Methyl paraben | 0.25 | 0.25 |
| 1 | Ethyl paraben | 0.15 | 0.15 |
| 1 | Propyl paraben | 0.10 | 0.10 |
| 1 | Butyl paraben | 0.05 | 0.05 |
| 1 | Trisodium EDTA | 0.05 | 0.05 |
| 1 | Cyclomethicone | QS100 | QS100 |
| 1 | Cyclomethicone, trimethylsiloxy silicate | 4.00 | 4.00 |
| 1 | Dimethicone | 10.00 | 10.00 |
| 1 | Polyglyceryl-4-isostearate, cetyl | 4.00 | 4.00 |
| | dimethicone copolyol, hexyl laurate | | |
| 2 | Tribehenin | 2.50 | 2.50 |
| 3 | Bentone Gel (cyclomethicone, disteardi- | 8.00 | 8.00 |
| | monium hectorite, denatured alcohol) | | |
| 4 | Isododecane | 5.00 | 5.00 |
| 4 | Tocopheryl acetate | 0.01 | 0.01 |
| 4 | Retinyl palmitate | 0.01 | 0.01 |
| 4 | Botanical mixture | 0.01 | 0.01 |
| 5 | Pigment Blend* | 4.06 | — |
| 5 | Pigment Blend** | — | 4.06 |

*40 parts galactoarabinan treated yellow iron oxide, 40 parts galactoarabinan treated red iron oxide, and 20 parts galactoarabinan treated black iron oxide, Color Techniques.
**40 parts yellow iron oxide (Sensient LCW Yellow iron oxide AQ), 40 parts red iron oxide (Sensient LCW red iron oxide AQ), and 20 parts black iron oxide (Sensient LCW black iron oxide AQ).

The composition was prepared by combining the Sequence 1 ingredients and grinding in a colloid mill until all the whites were dispersed and the pigment grind was uniform. The phase 1 ingredients were combined with the phase 2 waxes and heated to 75° C. The mixture was mixed with T-bar agitation and maintained at 70° C. The mixture was transferred to a homogenizer. The phase 3 ingredient was added and the mixture homogenized until uniform, while maintaining the temperature between 65 and 70° C. The phase 4 ingredients were added and the mixture cooled to 65° C. The mixture was transferred to the T-bar mixer and mixed on low speed agitation. The sequence 5 ingredients were added and mixed until no agglomerates were visible while maintaining the temperature about 55° C. The batch was poured into containers when cooled to a temperature of 55 to 60° C.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. An anhydrous color cosmetic composition comprising a pigment component and a powder component, the improvement wherein the powder component contains microfine particle size powders in an amount sufficient to provide a composition that will match more than one skin shade in one or more skin tone categories.

2. The composition of claim 1 wherein the pigment component comprises organic pigments, inorganic pigments, or mixtures thereof.

3. The composition of claim 2 wherein the pigment component comprises inorganic pigments.

4. The composition of claim 3 wherein the inorganic pigments are iron oxides.

5. The composition of claim 4 wherein the iron oxides have a particle size ranging from about 0.01-30 microns.

6. The composition of claim 4 wherein the iron oxides are red, yellow, black, or mixtures thereof.

7. The composition of claim 4 wherein the iron oxides are coated with an ingredient that makes the iron oxides hydrophilic.

8. The composition of claim 1 wherein the pigment comprises iron oxides coated with a hydrophilic ingredient, and are suspended in the oil phase of the emulsion when the composition is in the resting state.

9. The composition of claim 8 wherein the iron oxides are coated with one or more polysaccharides.

10. The composition of claim 8 wherein the polysaccharide comprises galactoarabinan.

11. The composition of claim 9 wherein the iron oxides are present ranging from about 0.01-75% by weight of the total composition.

12. The composition of claim 1 wherein the powder component comprises titanium dioxide.

13. The composition of claim 12 wherein a portion of the titanium dioxide has a particle size ranging from about 0.01-20 microns.

14. The composition of claim 13 wherein the microfine titanium dioxide is present in the composition in an amount ranging from about 0.5-15% by weight of the total composition.

15. The composition of claim 14 wherein the microfine titanium dioxide present in the composition comprises from about 50 to 95% of the total titanium dioxide content of the composition.

16. The composition of claim 1 wherein the anhydrous composition comprises reactive hydrophilic iron oxide pigments suspended in the oil phase of the composition.

17. The composition of claim 16 wherein the powder component comprises from about 0.01-70% by weight of the total composition, and wherein from about 50-95% of the total powder component comprises microfine powder particles having a particle size ranging from about 0.1-30 microns.

18. The composition of claim 17 where the composition has a first resting color and a second application color.

19. An anhydrous makeup composition having a first resting color and a second application color; comprising reactive pigments; wherein the reactive pigments suspended in the composition provide the first resting color, and upon application to skin provide the second application color.

20. The composition of claim 19 wherein the first resting color is non-skin-matching.

21. The composition of claim 20 wherein the non-skin-matching color is white, gray, or lightly tinted.

22. The composition of claim 20 wherein the second application color is skin-matching.

23. The composition of claim 22 which is a foundation makeup and wherein the skin matching color is a light, medium, or dark beige skin tone.

24. The composition of claim 19 wherein the reactive pigments have a particle size ranging from about 0.001-20 microns.

25. The composition of claim 24 wherein the reactive pigments are iron oxides.

26. The composition of claim 25 wherein the reactive pigments are coated with a material that facilitates their dispersion in the anhydrous composition.

27. The composition of claim 26 wherein the material is a polymer.

28. The composition of claim 27 polymer is a polysaccharide or silicone polymer.

29. An anhydrous color cosmetic composition comprising having reactive iron oxide pigments suspended therein.

30. The composition of claim 29, which is a foundation, blush, concealer, mascara, or lipcolor.
